(19) United States
(12) Patent Application Publication
(10) Pub. No.: US 2017/0016045 A1
(43) Pub. Date: Jan. 19, 2017

(54) SYSTEM AND METHOD FOR CONTAMINATION TESTING OF HIGH-TOUCH SURFACES

(71) Applicant: Level 11, LLC, Hayesville, NC (US)

(72) Inventor: Jefferson Scott McDaniel, Hayesville, NC (US)

(21) Appl. No.: 15/190,720

(22) Filed: Jun. 23, 2016

Related U.S. Application Data
(60) Provisional application No. 62/193,260, filed on Jul. 16, 2015.

(51) Int. Cl. C12Q 1/22; C12Q 1/04

(52) U.S. Cl. CPC C12Q 1/22; C12Q 1/04

(57) ABSTRACT

Methods and materials as may be utilized to provide consistent contamination testing of surfaces are described. The methods are based upon the designation of particular test zones on surfaces at risk of contamination by infectious agents. The designated test zone(s) can be tested and monitored over time and/or across like materials or devices for contamination. Templates for use in designating consistent test zones on high-touch surfaces are also described.

### CROSS REFERENCE TO RELATED APPLICATION

This application claims filing benefit of U.S. Provisional Patent Application Ser. No. 62/193,260, confirmation no. 1569, entitled “Test Square System For Contamination Testing,” having a filing date of Jul. 16, 2015, which is incorporated herein by reference in its entirety.

### BACKGROUND

In recent years, nosocomial disease due to secondary infection from bacterial, fungal, viral, or other pathogens has contributed a large part to increasing healthcare cost. In fact, hospital acquired infection (HAI) is estimated to cause upwards of $30 billion per year in excessive medical costs. In the acute care (e.g., hospital) market, it is estimated that 1 in every 25 patients may contract an HAI and an estimated 380,000 people die each year in long-term care facilities due to nosocomial infections. The most common nosocomial infections include bloodstream infection (BSI), pneumonia (eg, ventilator-associated pneumonia [VAP]), urinary tract infection (UTI), and surgical site infection (SSI).

A major cause of nosocomial infection is cross contamination during which bacteria or other pathogens are unintentionally transferred from one substance or object to another with harmful effect. In an attempt to decrease causes and sources of nosocomial infection such as cross contamination, various efforts have been made to better identify and track potential contamination locations and activities. For example, the Spaulding classification system proposed by Dr. E. H. Spaulding has been widely accepted and is used by the Food and Drug Administration (FDA), the Centers for Disease Control and Prevention (CDC), epidemiologists, microbiologists, and professional medical organizations. The Spaulding classification system divides medical devices into categories based on the risk of infection involved with their use to help determine the degree of disinfection or sterilization required for the devices.

The Spaulding risk levels associated with medical and surgical instruments include critical, semi-critical and noncritical. Critical items are defined as those items that normally enter sterile tissue or the vascular system, and items through which blood flows. Critical equipment must be sterile when used, based on one of several accepted sterilization procedures. Semi-critical items are defined as those objects that touch mucous membranes or skin that is not intact. Such items require meticulous cleaning followed by high-level disinfection treatment using an FDA-approved chemo-sterilizer agent. Non-critical items are defined as those that come into contact with intact skin or do not contact the subject. They require low-level disinfection by periodic cleaning and after visible soiling with a disinfectant detergent or germicide that is approved for healthcare settings.

In an attempt to decrease levels of HAI, the CDC has developed a two tiered program to optimize the thoroughness of high touch surface cleaning of non-critical items as part of terminal room cleaning at the time of discharge or transfer of patients. In order to obtain a high compliance rate with the higher Level II program, items must be tested and tracked according to recognized contamination monitoring systems such as by use of adenosine triphosphate (ATP) monitors or via test validation with cultures. Set guidelines are used to validate baseline numbers with broad testing and random sampling of 10-15% of surfaces on a periodic basis.

The CDC also recommends testing the same areas over time and among similar items in order that comparative data will be accurate and with scientific measure. Consistent comparison levels can assist in monitoring of high-touch areas and can provide information with regard to improvement of cleaning procedures among other benefits. Unfortunately, consistent testing remains a challenge, not only due to variation in testing techniques between item types, but also due to variations in testing techniques between personnel. For instance, after reviewing years of testing, it was determined that the test site on a given item type was rarely duplicated in the same exact area of the item.

In view of such issues, what are needed in the art are methods and systems for consistent contamination testing of high touch surfaces. Through improved consistency in contamination testing, for instance contamination testing of non-critical medical devices, improved data can be gathered with regard to effective cleaning methodologies as well as with regard to high-risk items and materials at increased risk of contributing to nosocomial infections.

### SUMMARY

According to one embodiment, a method for contamination testing a surface is disclosed. For instance, a method can include testing a designated test zone of a surface for contamination, storing a result of the testing, and repeating the testing of the designated test zone one or more times at a predetermined temporal interval. Through comparison of the results of the repeated testing procedures, information can be obtained regarding the risk of the surface for contamination by potential infectious agents, the effectiveness of cleaning procedures, and the like.

Also disclosed is a method for determining contamination levels of surfaces (e.g., high-touch surfaces) in a facility. For instance, a method can include testing a plurality of designated test zones. Each designated test zone can be on the same or different high-touch surfaces within a facility. The method can also include comparing the testing results obtained from the different test zones. Through comparison of the results, information can be obtained regarding relatively higher and lower contamination risk surfaces and/or areas in the facility. For instance, information can be obtained with regard to more or less effective cleaning protocols in different areas and/or on different items within a facility.

A template for contamination surface testing is also described. For instance, a template can include a border that surrounds an opening that can designate a contamination test zone. The border can include a first side and a second side. The first side can include markings, such as identification markings, cleaning instruction markings, and the like, and the second side can include an adhesive. During use, the template can be adhered to a surface and the opening of the template can designate a test zone on the surface.

These and other features, aspects and advantages of the present disclosure will become better understood with reference to the following description and appended claims.

### BRIEF DESCRIPTION OF THE DRAWINGS

A full and enabling disclosure of the present invention, including the best mode thereof, directed to one of ordinary skill in the art, is set forth in the specification, which makes reference to the appended figure, in which:

FIG. 1 is a schematic diagram of a surface including multiple contamination test zones on the surface.

FIG. 2 illustrates a template for use in defining a test zone on a surface.

FIG. 3 schematically illustrates one embodiment of a data interface as may be utilized in conjunction with disclosed contamination testing systems and methods.

### DETAILED DESCRIPTION

It is to be understood by one of ordinary skill in the art that the present discussion is a description of exemplary embodiments only, and is not intended as limiting the broader aspects of the present disclosure. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

In general, disclosed herein are methods and materials as may be utilized to provide consistent contamination testing of surfaces. More specifically, the methods and materials are based upon the designation of particular test zones on surfaces at risk of contamination by infectious agents. Once designated, the test zone(s) can be tested and monitored over time and/or across like materials or devices for contamination. For instance, multiple testing results obtained over time and/or from different surfaces can be collected in a database that can then be accessed and utilized to decrease nosocomial infection rates. By way of example, data can provide information with regard to effectiveness of cleaning protocols over time, for different materials, for different devices, by different cleaning personnel, in different areas of a single facility, or in different facilities.

In one particular embodiment, disclosed methods and materials can be utilized in contamination testing of high-touch surfaces of non-critical medical devices as defined by the Spaulding risk levels. For instance, disclosed methods and materials can be useful in contamination testing of surfaces of ventilator machines, blood pressure machines, imaging machines (e.g., ultrasounds, X-rays, CT machines, etc.), mattresses, feeding pumps, blower controllers, gurneys, curtains, data entry machines, delivery carts, heart monitors, etc. However, it should be understood that the methods and materials are in no way limited to such surfaces or to medical devices and/or medical care facilities.

The methods and materials can be utilized to assess contamination levels of any surface and in any location as may pose a risk of carrying infectious agents. By way of example, and without limitation, disclosed methods and materials can be utilized on high-touch surfaces in public facilities or private facilities such as hospitals, schools, hotels, restaurants, airports, entertainment arenas, etc. In addition, the methods and materials can be utilized on any natural or synthetic surface type, e.g., polymeric, metallic, ceramic, glass, wood, leather, textile, etc.

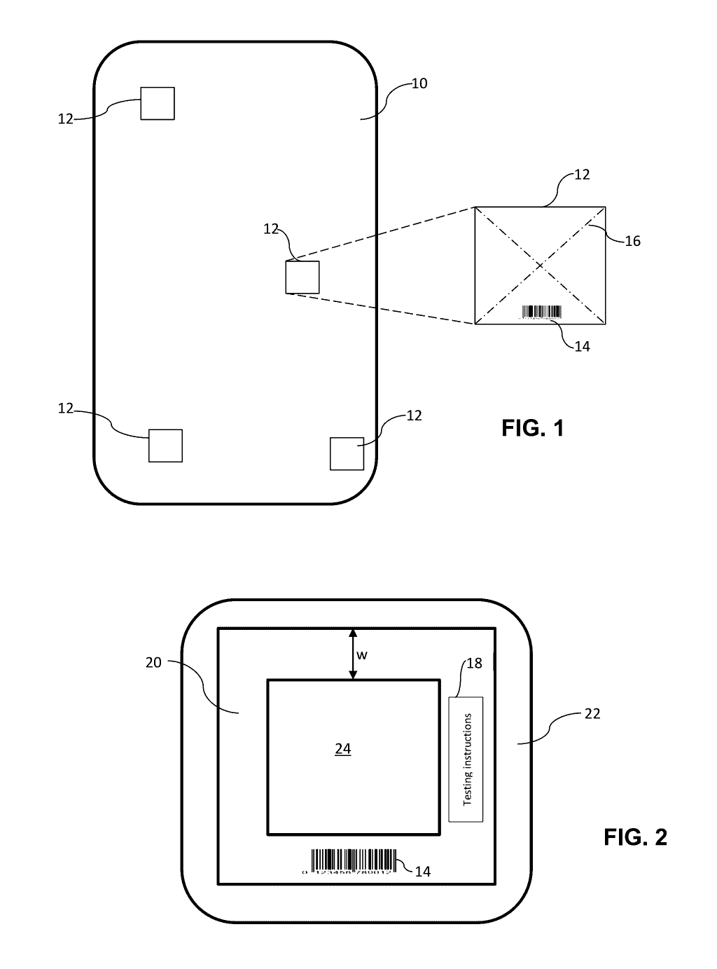

In one embodiment, a method can be utilized in testing non-critical devices in a medical care facility such as a hospital or long-term care facility. For example and as illustrated in FIG. 1, a surface of a hospital mattress 10 can include a plurality of designated test zones 12. While illustrated with four designated test zones 12, there is no particular requirement as to the number of test zones on any surface and a surface can include 1, 2, 3, 4, or more designated test zones on the surface. Moreover, a device can also or optionally include one or more designated test zones on other surfaces of the same device. For instance, a mattress can include designated test zones on one or more sides of the mattress as well as the bottom surface of the mattress.

A designated test zone 12 can be in a high-touch area that is at increased likelihood of contamination and/or cross contamination. For instance, the mattress 10 of FIG. 1 includes a test zone at the head of the mattress 10, which may be at more risk of cross contamination as compared to other areas of the mattress. Optionally, a surface can include a first test zone at a higher risk area of the surface and a second test zone at a lower risk area of the surface, which can provide comparative information following testing.

A test zone 12 can include markings that can provide information to a user. For example, FIG. 1 illustrates one of the test zones 12 in an expanded view. As shown, the test zone 12 includes an identifying mark 14 swab markings 16, and the like. In the illustrated embodiment, the identifying mark 14 is a bar code, but when included, any identifying mark is contemplated for a test zone such as, without limitation, a QR code, a trademark, an alphanumeric mark, a tactile mark, etc.

An identification mark can be utilized for identification purposes for the particular test zone as well as the item on which the test zone is located and the location of the item. For instance, in the illustrated embodiment, the identification mark 14 can designate the test zone, the particular mattress, the room in which the mattress is located, etc.

As a component of a testing protocol, the identification mark can be scanned or otherwise entered in a database and correlated with the testing of the zone 12. Thus, a record can be made of the date, time and location of a test done on the test zone 12.

The test zone 12 can also include markings that can be used during a testing protocol. For instance, in the illustrated embodiment, the test zone 12 includes swab markings 16 that can be used to encourage a user to fully swab the test zone 12 along the length of both of the markings 16.

Though illustrated as a square in FIG. 1, it should be understood that a test zone can be of any suitable size and shape. For instance, a test zone can be a polygon of any number of sides including without limitation a triangle, square, rectangle, etc. that can be regular or irregular; a circle, an oval, an ellipse, etc.

A test zone can be of a size so as to ensure sufficient surface area for obtaining an effectual examination of the zone for pathogenic contamination. In general, the test zone can be about 1 square inch (in²) (about 6.5 square centimeters (cm²)) or greater, such as about 10 in² or greater or about 15 in² or greater. For instance, a test zone can be from about 4 in² to about 16 in² (e.g., a 4 in.×4 in. square) in some embodiments.

Any suitable method and materials can be utilized to designate a test zone on a surface. The test zone designation should clearly delineate the area of the test zone and leave the surface material of the test zone uncovered so as to maintain the characteristic of the test zone surface and the surface of the device that surrounds the test zone as substantially identical. In other words, the test zone designation should be such that the surface within the test zone provides a representative example of the surface outside of the test zone.

When applying a test zone designation, any suitable designation style and type can be utilized. For instance, a test zone designation can be printed on a surface by use of, e.g., offset lithography, flexography, digital printing (inkjet, xerography, etc.), gravure, screen printing, etc.

A test zone can be formed on a surface during or following formation of the device upon which the zone is designated. For instance, a text zone designation can be printed or otherwise applied to a surface of a prior to or during formation of the device. For instance, a material for use in forming mattress covers, curtains, etc. can be formed to include test zone designations pre-printed on the surface. Accordingly, all products formed with the pre-printed materials can include the test zone designations with a consistent size and shape and in a consistent location on the final products.

In one embodiment, the test zone designation can be applied to a surface post-manufacture by use of a template that can be adhered to the surface. FIG. 2 illustrates one embodiment of a test zone designation template 20. As shown, the template 20 forms a border that surrounds an opening 24. The template 20 can generally have a width to provide suitable strength to the template and to clearly designate the test zone that will be surrounded by the template 20. For instance, the template 20 can have a width “w” of about 0.5 inches or greater, for instance from about 0.5 inches to about 2 inches in some embodiments.

During use, the template 20 can be adhered to a surface of a device and the portion of the device surface that is exposed in the opening 24 can be a contamination test zone. Though shown as surrounding a generally square opening 24, a template 20 can be of any size and shape and be utilized to designate a test zone of any size and shape as discussed above.

The template 20 can include identification markings 14, instructional markings 18, or any other markings on an upper surface that may be of benefit to the provider and/or the user (e.g., trademark, general information, website addresses, etc.).

As stated, a template 20 can be designed to be adhered to a surface so as to designate a contamination test zone within the opening 24. As such, a template can be formed of a robust material (i.e., a facestock) capable of remaining adhered to the surface of the tested device over time through expected uses, multiple cleanings, etc. For instance, a template facestock can include a layer of printable metal foil, paper/metal foil, MYLAR®, plastic (e.g., vinyl), or any other suitable material capable of forming to the surface of a device and withstanding expected operating conditions. A printable coating, such as an inkjet receptive coating, may be applied to the top surface so that the surface can be printed thereon. For purposes herein, it will be understand that the term “printing” includes writing by pen or ink, typewriting, laser printing, ink jet printing, or any other manner of placing text or graphics thereon.

In one embodiment, the template 20 can be provided with a section of the facestock material in the opening 24. The template 20 can be pulled or peeled away from this adjacent facestock section when the template is removed from the liner sheet 22 for placement on a surface. This additional facestock can likewise be printed with any information or markings as desired. This section will generally be simply disposed of with the liner sheet following placement of the template 20 on the desired surface.

A template 20 can be provided in conjunction with a liner sheet 22 that can hold and support the template 20 prior to adhesion of the template 20 to the desired testing surface. The template 20 can be releasably adhered to the liner sheet 22 by an adhesive, e.g., a pressure sensitive adhesive as is well known. The pressure sensitive adhesive on the back of the template 20 is usually, though not necessarily, of sufficient strength as to permanently adhere the template 20 to a surface after the template has been peeled from the liner sheet and applied to the object. Such a pressure sensitive adhesive is generally referred to as a permanent pressure sensitive adhesive, as opposed to a releasable pressure sensitive adhesive. Even though the pressure sensitive adhesive is often of the permanent type, the template can be easily removed from the liner 22 because the liner 22, often referred to as the release liner, is covered with a non-stick coating such as a thin layer of silicone.

The template 20 can optionally include holding tabs or “no touch tabs” (not illustrated in FIG. 2), so that the user can hold the template 20 while it is being positioned over the testing surface for application thereon without touching an adhesive surface. The holding tabs are typically removed from the template 20 after the template has been placed onto the surface by tearing along perforations or cuts and ties formed in the template.

Beneficially, the utilization of an adhesive template can provide for the designation of test zones in a consistent fashion throughout a facility. For instance, all mattresses of a single hall, ward, or hospital, can have test zones designated in the same locations on the mattresses. Similarly, all ventilators, imaging devices, etc. can be consistently marked throughout a facility.

During use, the designated contamination testing zone can provide a consistent area for surface testing. Any surface testing methodology can be carried out as is generally known in the art including, without limitation, swab cultures, agar slide cultures, fluorescent markers, and ATP or other bioluminescence testing.

The testing information including the specific test zone, the time and place of the test and the test results can be correlated in a database that can then be used to provide information with regard to the cleanliness of the surfaces, the devices, the facility, etc. For instance, the data from multiple tests at a single testing zone taken at different times can be examined to determine cleanliness information of that particular surface (e.g., are different cleaning protocols being utilized at different times, if so are some more effective than others, is this device showing increased contamination likelihood over time—is replacement of the device necessary, etc.)

A system can also be utilized to examine the effectiveness of cleaning protocols throughout a facility through comparison of the testing results of similar devices. For instance, the surface testing results of similarly located testing zones on all mattresses (or any other device to be tested) can be compared to one another to determine if cleaning protocols are equivalent across a facility as well as across time. Differences between test results can be utilized to examine differences in cleaning protocols used in different departments, on different shifts, on different devices, etc.

A data system can be utilized to compile and track specific equipment categories at highest risk of contamination and compare cleaning effectiveness both over time and among different personnel and cleaning protocols and can provide an ongoing validation system to monitor cleaning effectiveness.

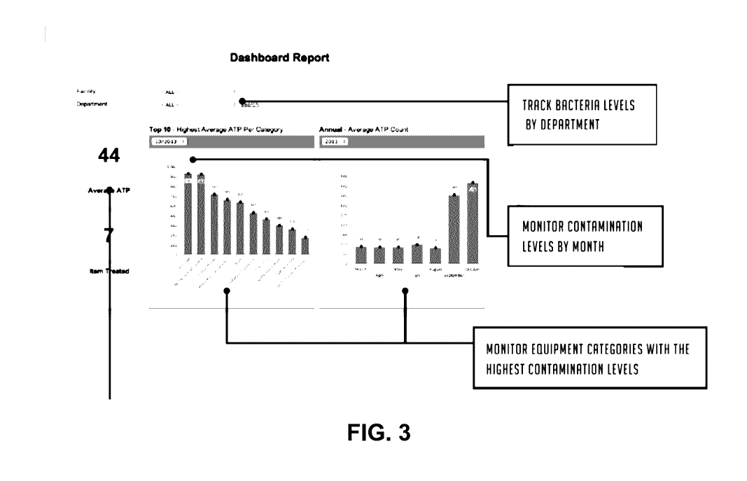

By way of example, and without limitation, the compiled data can be accessible via a software system such as a dashboard system as is known in the art. One example, of a dashboard data delivery system is illustrated in FIG. 3. Essentially, a data compilation and delivery system can provide for testing data to be transferred to and compiled in an external device, e.g., a computer. For instance, upon carrying out a surface test at a designated test zone, identification marks of the test zone (e.g., a bar code) can be read and transferred to an external device via e.g., direct, wireless, internet connection or other communications methodologies as may be available or made available. The time and date of the test can also be recorded and transferred to the external device. Depending upon the type of surface test that is carried out at the testing zone, the results may be immediately available and thus able to be transferred to the external device at the time of carrying out the surface test, or alternatively, may be made available at a later time. For instance, if the surface at the testing zone is swabbed, this information can be recorded and the swab marked (e.g., by a label) so as to correlate the particular swab with the test zone as well as with the time and date of the test. When the test results are later available (e.g., following culturing of the swab), these test results can then also be provided to the external device and correlated with the test zone, time and date of the test.

An external device may be accessible to only the user of device or may be a shared device. For instance, an external device may be a private computer, within the office of the responsible party (e.g., a surface testing company, a hospital division, etc.), or may widely accessible at a monitoring facility, for instance throughout a medical facility, such that appropriate medical personal may be informed of the testing results at any time.

Upon receipt of data at the external device, the data may be converted to sequential user records and stored in a relational database format (RDBMS) where at least a User ID, Mode, and Date/Time of Measurement may be used as primary keys along with a User's unique ID (e.g., as maintained in the RFID chip 360).

As illustrated in FIG. 3, the data may be accessible in any desired dashboard-type format. For instance, in the illustrated embodiment, departments can be separately monitored for overall contamination levels or for contamination levels over time; contamination levels throughout an entire facility can be monitored over time; contamination levels for various equipment types can be monitored for differences over time and/or for differences between areas of a facility; etc.

Any database management and/or access protocols as are generally known in the art can be utilized to provide extensive information with regard to the contamination levels of the equipment. Through utilization of consistent contamination testing zones both on a single device over time and across multiple of the same types of devices, the acquired data can be more accurate and more useful in providing real-world information so as to improve and decrease cross contamination and nosocomial infection rates.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A method for contamination testing a surface, the method comprising:
testing a designated test zone on the surface for contamination;
storing a result of the testing; and
repeating the testing of the designated test zone one or more times at an interval.

2. The method of claim 1, further comprising comparing test results from the repeated testing.

3. The method of claim 1, wherein the surface is the surface of a medical device.

4. The method of claim 3, wherein the medical device is a non-critical medical device as defined by the Spaulding risk levels.

5. The method of claim 1, further comprising testing one or more additional designated test zones on the surface.

6. The method of claim 1, wherein the step of storing the result of the testing further comprises correlating the test result with the designated test zone and with a time and date of the test.

7. The method of claim 1, wherein the designated test zone is designated by a printed marking on the surface.

8. The method of claim 1, wherein the designated test zone is designated by a template adhered to the surface.

9. A method for determining contamination levels in a facility comprising:
testing a plurality of designated test zones for contamination, each of the designated test zones being on different surfaces throughout a facility;
storing results of the testing; and
comparing the test results.

10. The method of claim 9, further comprising repeating the testing at predetermined intervals.

11. The method of claim 9, wherein the different surfaces are on the same type of device throughout the facility.

12. The method of claim 9, wherein the different surfaces are on different types of devices throughout the facility.

13. The method of claim 9, wherein the designated test zones are designated by a printed marking on the surfaces.

14. The method of claim 9, wherein the designated test zones are designated by a template adhered to the surfaces.

15. The method of claim 9, wherein the facility is a medical facility or a long-term care facility.

16. A template for contamination surface testing, the template comprising a border surrounding an opening, the opening designating a test zone, the template comprising an upper surface and a lower surface, the lower surface including an adhesive.

17. The template of claim 16, the upper surface comprising an identification marking.

18. The template of claim 16, wherein the adhesive is a pressure sensitive adhesive.

19. The template of claim 16, further comprising a liner sheet.

20. The template of claim 16, wherein the opening has an area of about 1 square inch or greater.
